(19) United States
(12) Patent Application Publication
(10) Pub. No.: US 2024/0181190 A1
(43) Pub. Date: Jun. 6, 2024

(54) INTUBATION ASSIST DEVICE

(71) Applicant: Elicore Medical Solutions, LLC, Johnston, RI (US)

(72) Inventors: Vito Ferri, Cranston, RI (US); Christopher Ferri, Cranston, RI (US); Richard Pedro, Assonet, MA (US); Louis Insalaco, Wilmington, MA (US); Ryan Murphy, West Orange, NJ (US); Cary Vachon, Schenectady, NY (US)

(21) Appl. No.: 18/441,897

(22) Filed: Feb. 14, 2024

Related U.S. Application Data
(63) Continuation-in-part of application No. 16/725,328, filed on Dec. 23, 2019.

(51) Int. Cl. A61M 16/04; A61M 1/00; A61M 25/01

(52) U.S. Cl. CPC A61M 16/0463; A61M 1/7411; A61M 1/76; A61M 1/86; A61M 16/0488; A61M 25/0102; A61M 2205/3306; A61M 2205/586; A61M 2205/587

(57) ABSTRACT

Embodiments of the present invention disclose a suction stylet assembly for endotracheal intubation. The assembly includes an elongated tubular member, the elongated tubular member having one or more apertures proximate to a first end, and a handle in communication with a second end of the elongated tubular member. The handle member includes a first passageway connecting the elongated tubular member to a hose fitting on the handle member, the hose fitting configured to receive a hose that provides suction in removing bodily fluids. The handle member further includes an aperture located on a side of the handle member, the aperture forming a second passageway through the handle member running between the side and the first passageway, the aperture configured to permit a user to selectively block the aperture, thereby selectively controlling suction provided between the hose fitting and the elongated tubular member.

### CROSS REFERENCE TO RELATED APPLICATIONS

This application is a Continuation in Part of U.S. patent application Ser. No. 16/725,328, filed on Dec. 23, 2019, the disclosure of which is incorporated by reference herein in its entirety.

### FIELD OF THE INVENTION

The present application relates to endotracheal tubes, and more particularly to technologically advanced stylets that guide endotracheal tubes for intubation.

### BACKGROUND OF THE INVENTION

A common procedure performed by medical professionals to assist a patient to breathe is intubation through the use of an endotracheal tube. This may be performed during surgery or during prolonged need for breathing assistance, such as a stay in an intensive care unit. A standard endotracheal tube has a cuff located proximately at a first end of the tube to seal the end of the tube against the inner walls of a trachea where a pilot balloon connects to the cuff to inflate the cuff. During a standard intubation, the medical professional will commonly place a malleable stylet inside the endotracheal tube and then bend the endotracheal tube and stylet together to a desired shape to optimize intubation. Once the endotracheal tube is in the desired location in the trachea, the stylet is removed, and the endotracheal tube is left in place in the trachea. The cuff is then inflated, and a ventilator circuit is attached to a connector located at a second end of the tube.

During intubation, the medical professional that is intubating the patient uses one hand to expose an opening to the patient's windpipe while the professional's second hand maneuvers the endotracheal tube. Often during this process, the medical professional must use their second hand to suction mucous, blood, or vomit from the patient's throat in order to obtain an adequate view of the vocal cords, between which the endotracheal tube is placed. However, to remove these fluids, the professional must set aside the endotracheal tube to use a suction device, which can waste valuable time and allow for re-accumulation of obstructing fluids and debris in often crucial periods where the patient's airway is not secured. Therefore, it is desired to develop a device that provides for simultaneous suction and intubation without having to use a separate suction device.

### SUMMARY OF THE INVENTION

Embodiments of the present invention disclose a suction stylet assembly for endotracheal intubation. In one embodiment of the present invention, an assembly is provided comprising: an elongated tubular member having a first end and a second end, the elongated tubular member having one or more apertures proximate to the first end; a handle member having a first end, a second end, and a side that runs between the first end and the second end, the first end of the handle member in communication with the second end of the elongated tubular member, the handle member including: a first passageway passing through the handle member running from the first end to the second end, the elongated tubular member coaxial with the first passageway; a hose fitting located at the second end, the hose fitting configured to receive a hose that provides suction in removing bodily fluids; and an aperture located on the side, the aperture forming a second passageway through the handle member running between the side and the first passageway, the aperture configured to permit a user to selectively block the aperture with a digit of the user, thereby selectively controlling suction provided between the hose fitting of the handle member and the first end of the elongated tubular member.

Ultimately the invention may take many embodiments. In these ways, the present invention overcomes the disadvantages inherent in the prior art.

The more important features have thus been outlined in order that the more detailed description that follows may be better understood and to ensure that the present contribution to the art is appreciated. Additional features will be described hereinafter and will form the subject matter of the claims that follow.

Many objects of the present application will appear from the following description and appended claims, reference being made to the accompanying drawings forming a part of this specification wherein like reference characters designate corresponding parts in the several views.

Before explaining at least one embodiment of the present invention in detail, it is to be understood that the embodiments are not limited in its application to the details of construction and the arrangements of the components set forth in the following description or illustrated in the drawings. The embodiments are capable of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods, and systems for carrying out the various purposes of the present design. It is important, therefore, that the claims be regarded as including such equivalent constructions in so far as they do not depart from the spirit and scope of the present application.

### BRIEF DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a side view of a stylet assembly having a sheath portion for coupling with a connector of an endotracheal tube, in accordance with an embodiment of the present invention.

FIG. 2 is a top view of the stylet assembly of FIG. 1.

FIG. 3 is a side view of an endotracheal tube having a connector.

FIG. 4 is a top view of the endotracheal tube of FIG. 3.

FIG. 5 is a side view of the stylet assembly of FIG. 1 coupled with the endotracheal tube of FIG. 3.

FIG. 6 is a top view of the stylet assembly and endotracheal tube of FIG. 5.

FIG. 7 is a side view of a stylet assembly having a tapered portion for coupling with a connector of an endotracheal tube.

FIG. 8 is a side view of the style assembly of FIG. 7 coupled with the endotracheal tube of FIG. 3.

FIG. 9 is a view of the suction stylet assembly in position in a patient.

FIG. 10A is a top view of a prototype suction stylet assembly with optional example features 575 and 580 at the distal (or insertion end). FIG. 10B is an enlarged top view of the handle from the prototype suction stylet assembly of FIG. 10A. FIG. 10C is an enlarged top view of the distal (insertion) end of the prototype suction stylet assembly of FIG. 10A, with some examples of optional features 575 and 580 shown as a camera 575, light 581, and others. FIG. 10D is a side view of the prototype suction stylet assembly of FIG. 10A. showing optional actuators 596, 597 to extend control 575 and/or 580. FIG. 10E is an enlarged side view of the handle from the prototype suction stylet assembly of FIG. 10D.

FIG. 11A shows a prototype suction stylet assembly in an engaged position with an endotracheal tube (or intubation tube) in a pre-bent (straight) shape. FIG. 11B shows the prototype suction stylet assembly from FIG. 11A in a semi-bent shape, with the endotracheal tube or intubation tube, during preparation for insertion into the patient. FIG. 11C shows two enlarged views of the tip or distal end of the prototype suction stylet assembly in an engaged position with the tip or insertion end of an endotracheal tube (or intubation tube) highlighting example positions of the features in FIG. 10C. FIG. 11D shows an enlarged view of the tip or distal end of the prototype suction stylet assembly in an engaged position with the tip or insertion end of an endotracheal tube (or intubation tube) and different configurations for the optionally extendable features 575 and 580 from the examples in FIG. 10C.

FIG. 12A (prior art) shows a typical (older past technology) method for intubation along with suction taking 8 steps and about 60-90 seconds while the patient's precious seconds are at stake. FIG. 12B shows an advanced method for intubation along with suction (that is enabled by the presently disclosed technology) and along with other optional features exemplified in FIG. 10C, taking 4 steps and about ≤30 seconds in consideration of the patient's precious seconds.

FIG. 13A shows a photograph from an observer's eye view or a nurse's eye view of a doctor (or healthcare provider) using a system of the present technology to simultaneously maneuver, suction, and intubate using a single hand 1000 and finger 405 (or digit) controlling suction during this simultaneous work. The maneuverable handle 507 of the stylet allows a close proximity with the other hand 1005 that is providing a laryngoscope and other necessary functions. FIG. 13B shows a photograph from the doctor's (or surgeon's) eye view while using a system of the present technology to simultaneously manipulate, video, suction, and intubate with a single hand 1000 and finger 405 controlling suction, while the other hand 1005 is providing a light and laryngoscope. FIG. 13C shows a photo from the same doctor's eye view of FIG. 13B of the digital display 1500 of the video camera that is inside the patient 400, highlighting how the single hand 1000 can provide suction and intubation while the doctor sees what is happening at the distal end.

FIG. 14A shows a photo of a prototype handle of the current technology highlighting the maneuverable, single-hand control handle. FIG. 14B shows a photo of a prototype stylet tube of the current technology highlighting the optimum bendability enabled in conjunction with the handle in FIG. 14A. FIG. 14C shows a photo of a prototype distal end of a stylet of the current technology highlighting integration with an endotracheal tube (intubation tube) along with optional features at the end of the stylet.

FIG. 15A shows a diagram of a prototype (clear, coated, or painted) handle of the current technology, highlighting the clear material that is transparent (e.g., polycarbonate or other material) option or opaque option along with an example aluminum material stylet tube. The materials used herein can vary and can be any suitable material known in the art. Although not shown, one or more coatings can be utilized on any of the aspects. FIG. 15B shows a diagram of a prototype handle of the current technology. Any suitable materials can be utilized, for example, a PC-ABS (polycarbonate/acrylonitrile-butadiene-styrene terpolymer blend) using a thermoplastic alloy of (PC) polycarbonate and (ABS) acrylonitrile-butadiene-styrene prototype construction is illustrated. FIG. 15C shows mechanical diagrams illustrating example test dimensions of a prototype the current technology highlighting the testing of (single-handed) maneuverability provided by the handle dimensions. The materials used can vary and the examples in FIG. 15A, FIG. 15B, and FIG. 15C are examples. Other suitable materials known in the art can be utilized.

While the embodiments and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the embodiments described herein may be oriented in any desired direction.

The assembly and method in accordance with the present invention overcomes one or more of the above-discussed problems associated with endotracheal tubes during patient intubation when patient bodily fluids (e.g., mucous, blood, vomit, etc.) occlude breathing passageways to the patient's trachea. In particular, the system of the present invention is a suction stylet having a malleable elongated tubular member and a handle member. The handle member is located at one end of the tubular member and has a first passageway running through the handle member that connects from the tubular member to a hose fitting on the handle member. Furthermore, the handle member has a handle aperture located on a side of the handle member, the aperture being an opening to a second passageway that connects between the side and the first passageway. The handle aperture and associated second passageway permits selective control of suction between a hose fitting (connected to a suction source) and a second end of the tubular member: when the handle aperture is in an open state, suction provided to the hose fitting draws ambient air through the second passageway to the first passageway, thereby redirecting suction away from the tubular member. A user operating the suction stylet assembly directs suction to the tubular member to remove unwanted bodily fluids during intubation by blocking the handle aperture, thereby directing suction from the hose fitting to the tubular member. In some embodiments, multiple side apertures to help allow for variable degrees of suction to be applied are implemented (instead of a single side aperture). In this example, there can be 2 or more, or a plurality of side apertures, and the user uses a finger (or thumb depending on the user) to cover up more apertures to apply more suction at the tip. In this precision feature, a plurality of smaller side apertures can provide a higher degree of precision in the vacuum control. In some embodiments, the number of side apertures can be in the range from about 2-50, in the range from about 2-25, in the range from about 2-20, in the range from about 2-10, or optionally in the range from about 2-5 (i.e., all endpoints throughout this disclosure are subsumed).

The elongated tubular member of the suction stylet is configured to slide into an endotracheal tube through a connector located at one end of the endotracheal tube. The handle member of the suction stylet serves as a stop against the connector to restrict the suction stylet from further insertion into the endotracheal tube. The assembly may come in various sizes to correspond to various endotracheal tube sizes. Furthermore, the elongated tubular member may have a murphy eye that concentrically aligns with a murphy eye typically found on endotracheal tubes. Through the combination of the features listed above, the suction stylet thus allows for simultaneous suction and intubation.

The system will be understood from the accompanying drawings, taken in conjunction with the accompanying description. Several embodiments of the system may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

The system of the present application is illustrated in the associated drawings. As used herein, “system” and “assembly” are used interchangeably. As used herein, a “fastener” is a rod-like hardware device that mechanically joins or affixes two or more members together through a respective concentric set of apertures. For example, a fastener can be a screw, bolt, nail, stud, dowel, rivet, staple, etc. in conjunction with any applicable nuts and washers generally known in the art of fastening. It should be noted that the articles “a”, “an”, and “the”, as used in this specification, include plural referents unless the content clearly dictates otherwise. Additional features and functions are illustrated and discussed below.

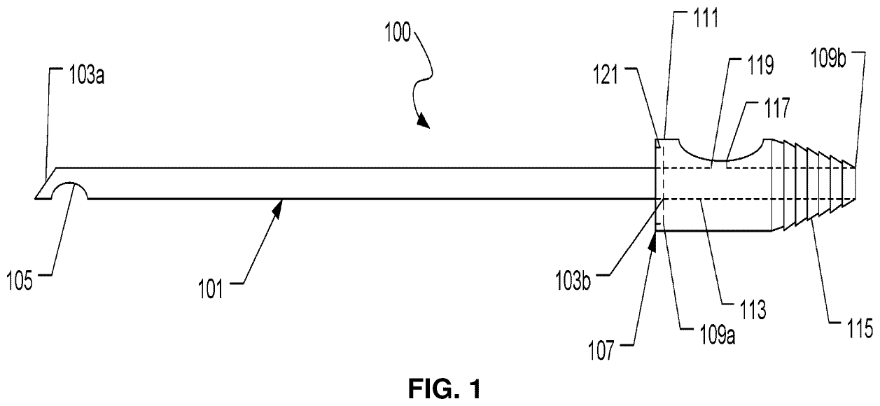

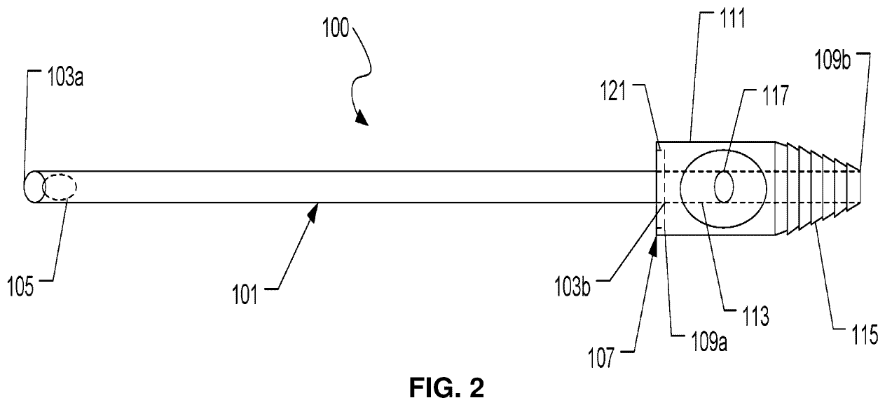

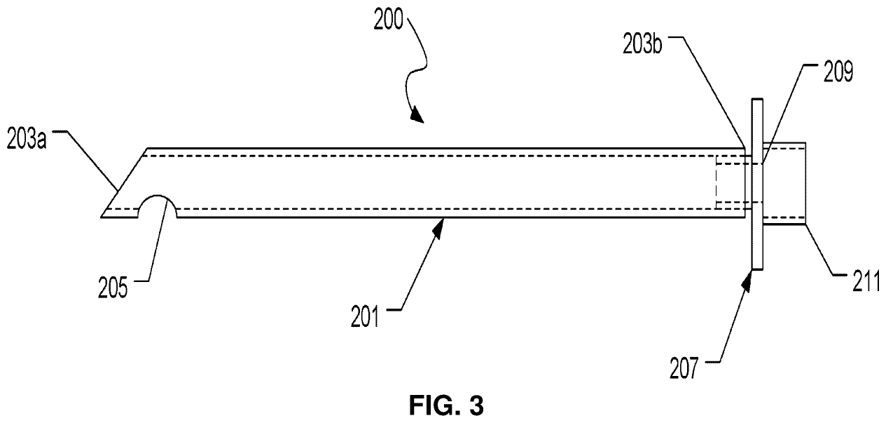

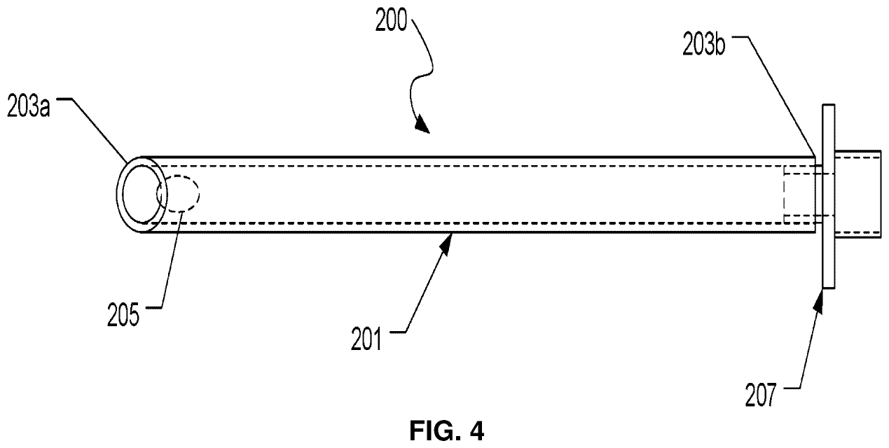

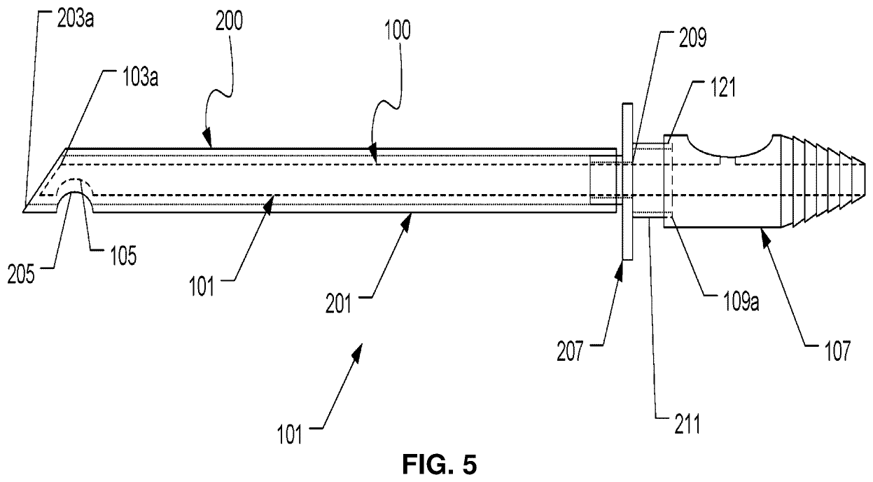

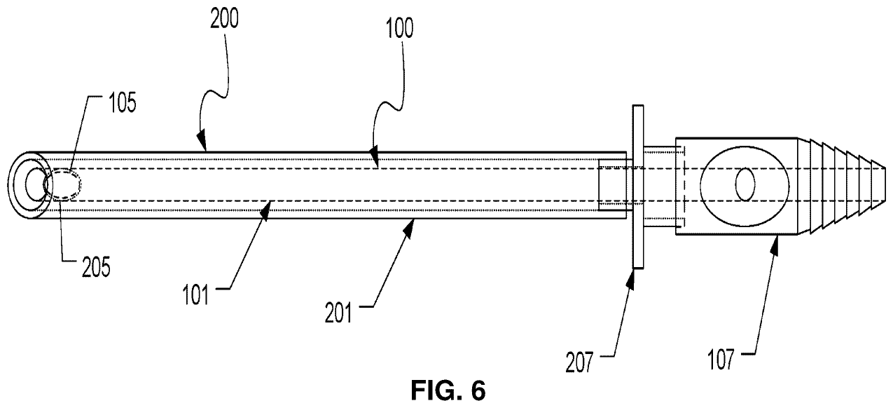

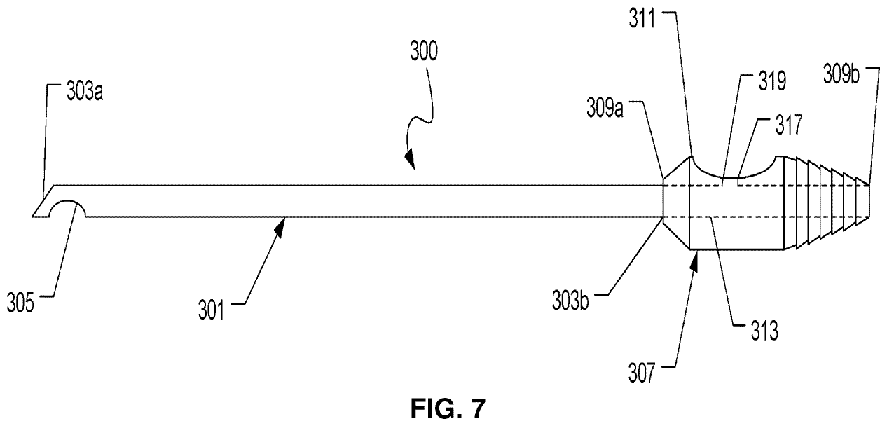

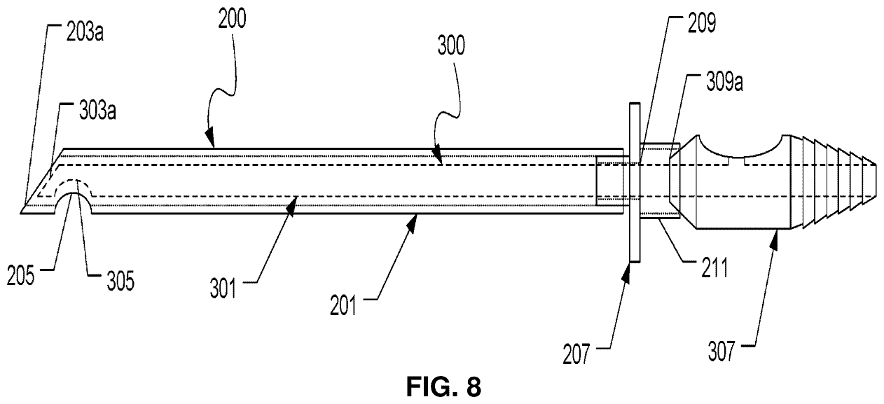

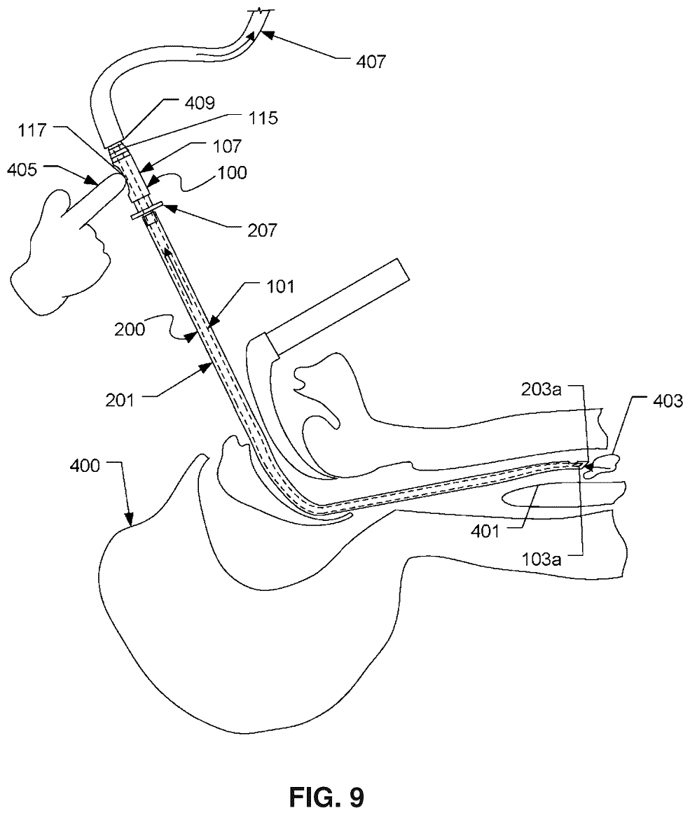

Referring now to the drawings wherein like reference characters identify corresponding or similar elements in form and function throughout the several views. FIGS. 1-2 illustrate various views of a stylet assembly having a sheath portion for coupling with a connector of an endotracheal tube. FIGS. 3-4 illustrate various views of an endotracheal tube generally known in the art. FIGS. 5-6 illustrate various views of the stylet assembly coupled with the endotracheal tube. FIGS. 7-8 illustrate various views of a stylet assembly having a tapered portion for coupling with a connector of an endotracheal tube. FIG. 9 is a view of the suction stylet assembly in position in a patient.

Referring now to FIG. 1, a top view of stylet assembly 100 is illustrated in accordance with an embodiment of the present invention.

In FIG. 1, stylet assembly 100 includes, but is not limited to, elongated tubular member 101 and handle member 107. In general stylet assembly 100 is a suction stylet that serves as a stylet in providing structural support for an endotracheal tube during intubation of a patient as well as providing a suction device for removing unwanted bodily fluids that occlude breathing passages of the patient.

Elongated tubular member 101 is a malleable metal tube. In general, elongated tubular member 101 may be bent by a user into a configuration suitable for patient intubation, wherein once the tubular member 101 is bent into the configuration, tubular member 101 provides structural support for an endotracheal tube during intubation. Optionally, a flexible plastic rod may be inserted into tubular member 101. The flexible plastic rod and elongated tubular member 101 may be bent together into a configuration suitable for patient intubation, wherein the flexible plastic rod prevents tubular member 101 from kinking while the flexible plastic rod and tubular member 101 are bent into the configuration.

Furthermore, tubular member 101 serves as a conduit for removing unwanted bodily fluids located proximate to a first end to a second end via air suction. Optionally, the elongated tubular member 101 may be coated with a soft synthetic material to allow for low friction removal from an endotracheal tube once patient intubation is successful. Tubular member 101 has ends 103a and 103b having corresponding openings at each end. As used herein, end 103a is a distal end of stylet assembly 100. In some instances, end 103a is a beveled end. Optionally, tubular member 101 has one or more apertures proximate to end 103a. For example, in FIG. 1, aperture 105 is located proximate to end 103a, wherein aperture 105 is a murphy eye similar in form and function as murphy eyes commonly found in the field of endotracheal tubes. However, it should be understood that the one or more apertures, such as aperture 105, may have variable sizes or may be omitted entirely.

Handle member 107 is a handle having ends 109a and 109b. In general, handle member 107 is gripped by a user during intubation, during removal of stylet assembly 100 from an endotracheal tube, and/or during suction of unwanted bodily fluids of a patient; the user being able to selectively control suction at end 103a by selectively blocking an aperture located on the handle associated with a passageway that diverts suction from end 103a to ambient air located externally from the patient. Handle member 107 has side 111 that runs between ends 109a and 109b. End 109a of handle member 107 is in communication with end 103b of tubular member 101. In some instances, handle member 107 is plastic.

Handle member 107 includes, but is not limited to, a first passageway (i.e., passageway 113), a hose fitting (i.e., hose fitting 115), and a side aperture (i.e., aperture 117) associated with a second passageway (i.e., passageway 119). In this FIG. 1, passageway 113 passes through handle member 107 running from ends 109a and 109b with tubular member 101 being coaxial with passageway 113. Hose fitting 115 is located at end 109b, wherein hose fitting 115 is a hose fitting configured to receive a hose that provides suction in removing bodily fluids. For example, hose fitting 115 can be a Christmas tree hose fitting as illustrated in FIG. 1. However, it should be understood that a variety of alternate hose fittings may be employed for receiving suction hosing at end 109b.

In this FIG. 1, side aperture 117 is located on side 111. Side aperture 117 forms passageway 119 through handle member 107 that runs between side 111 and passageway 113. In this FIG. 1, side aperture 117 is configured to permit a user to selectively block aperture 117 with a digit of the user, thereby selectively controlling suction provided between hose fitting 115 and end 103a of tubular member 101.

Furthermore, end 109a of handle member 107 serves as a stop against a connector of an endotracheal tube to restrict stylet assembly 100 further insertion into the endotracheal tube. End 109a may come in a variety of configurations to form a stop. For example, end 109a may have a portion formed as a female connection for receiving the connector of an endotracheal tube as a male connector, or end 109a may have a tapered portion that forms a male connector configured to receive the connector of the endotracheal tube as a female connector. In FIG. 1, handle member 107 has end 109a recessed into handle member 107 to form a sheath portion (i.e., sheath 121) as a female connector that is configured to receive a connector of an endotracheal tube as a male connector. End 109a formed as a male connector is further illustrated and described with respect to FIGS. 7 and 8 (see end 309a)

Referring now to FIG. 2, a top view of stylet assembly 100 of FIG. 1 is illustrated.

Referring now to FIGS. 3 and 4, a side view and top view of endotracheal tube assembly 200 having a connector are illustrated respectively.

In these FIGS. 3 and 4, endotracheal tube assembly 200 is a typical endotracheal tube known in the art having a connector located at one end of the tube. While some endotracheal tubes include a balloon cuff located proximate to an end of the tube that is inserted into a trachea of a patient, the balloon cuff feature has been omitted from the drawings for simplicity. In these FIGS. 3 and 4, endotracheal tube 201 has ends 203a and 203b, wherein end 203a has a beveled end, a murphy eye (i.e., murphy eye 205) is located proximate to end 203a, and connector 207 is in communication with end 203b. In general, connector 207 is a universal or standard endotracheal tube connector that allows for connection to a ventilator circuit once the endotracheal tube is inserted into a trachea of patient. In these FIGS. 3 and 4, connector 207 has aperture 209 and cuff 211, wherein aperture 209 permits air to pass through connector 207, and cuff 211 is a portion of connector 207 that connects to a ventilator circuit.

Referring now to FIGS. 5 and 6, a side view and a top view of stylet assembly 100 coupled with endotracheal tube assembly 200 are illustrated respectively.

In these FIGS. 5 and 6, stylet assembly 100 is coupled with endotracheal tube assembly 200 by inserting end 103a of stylet assembly 100 through aperture 209 of endotracheal tube assembly 200 until connector 211 is in contact with end 109a of handle member 107. In this FIG. 5, sheath 121 sheaths an end of connector 211. In this configuration, tubular member 101 is sheathed by endotracheal tube assembly 200. Furthermore, a user aligns stylet assembly 100 with endotracheal tube assembly 200 such that murphy eye 105 of stylet assembly 100 is concentric with murphy eye 205 of endotracheal tube assembly 200.

Referring now to FIG. 7, a side view of stylet assembly 300 having a tapered portion for coupling with connector 207 of endotracheal tube assembly 200 is illustrated.

In this FIG. 7, stylet assembly 300 has the same form and function as stylet assembly 100 except for end 309a being a tapered portion of the handle member instead of having sheath 121. In other words, the following components have similar form and function: elongated tubular member 101 is similar to elongated tubular member 301; end 103a is similar to end 303a; end 103b is similar to end 303b; murphy eye similar to murphy eye 305; handle member 107 is similar to handle member 307; end 109b similar to end 309b; side 111 is similar to side 311; passageway 113 is similar to passageway 313; aperture 117 is similar to aperture 317; and passageway 119 is similar to passageway 319. In this FIG. 7, end 309a is a tapered portion that forms a male connector configured to receive a connector of an endotracheal tube as a female connector.

Referring now to FIG. 8, a side view of stylet assembly 300 coupled with endotracheal tube assembly 200 is illustrated. In this FIG. 8, stylet assembly 300 is coupled with endotracheal tube assembly 200 by inserting end 303a of stylet assembly 300 through aperture 209 of endotracheal tube assembly 200 until connector 211 is in contact with tapered end 309a of handle member 307.

Referring now to FIG. 9, a view of stylet assembly 100 and endotracheal tube assembly 200 in position in a patient. In this FIG. 9, a medical professional inserts elongated tubular member 101 of stylet assembly 100 into endotracheal tube assembly 200 through connector 207. The professional then positions handle member 107 in contact with connector 207 and connects end 409 of suction tubing 407 to hose fitting 115, wherein suction tubing 407 provides suction for removing unwanted bodily fluids. The professional then bends tubular member 101 and endotracheal tube 201 into a configuration optimal for intubation, wherein tubular member 101 structurally maintains the bent configuration for endotracheal tube 201. The professional then inserts end 103a of stylet assembly 100 and end 203a of endotracheal tube assembly 200 into trachea 401 of patient 400. To remove fluids 403 that is occluding trachea 401, the professional selectively blocks side aperture 117 on handle member 107 to selectively provide suction to end 103a of tubular member 101 provided by the suction tubing 407, wherein selectively blocking side aperture 117 is performed with digit 405 of the professional. Blocking side aperture 117 provides suction that draws fluids 403 located proximate to end 103a through tubular member 101 and handle member 107, and to connected suction tubing 407. Not blocking side aperture 117 results in ambient air being drawn through aperture 117 and to connected suction tubing 407, thereby diverting suction away from end 103a. Once endotracheal tube assembly 200 is in position in trachea 401 and fluids 403 are removed, the professional removes stylet assembly 100 from endotracheal tube assembly 200.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

In emergency situations, with about 50 million intubations happening yearly worldwide, saving precious seconds can mean the difference between life and death of a loved one. With the large numbers of intubations, the technology disclosed herein saves those critical seconds that add up to many saved lives. From a hectic emergency room to a motor vehicle accident site, the technology provides 2-in-1, 3-in-1, 4-in-1, 5-in-1 or even greater functionalities enabled in a single hand operable life-saving stylet. The devices are composed of FDA-approved (US Food and Drug Administration approved) materials that are ergonomically designed in shapes that allow close contact with the other hand, with the patient, and with other emergency devices providing multiple functions during said close contacts.

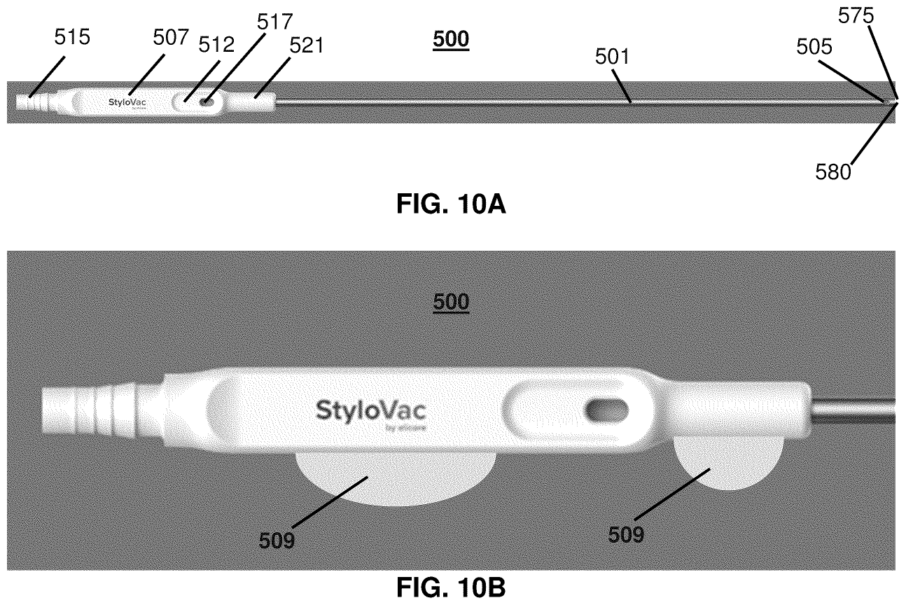

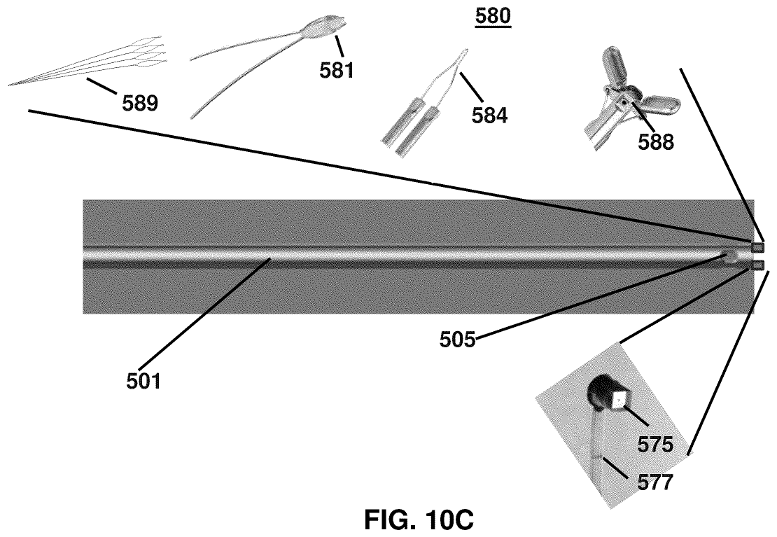

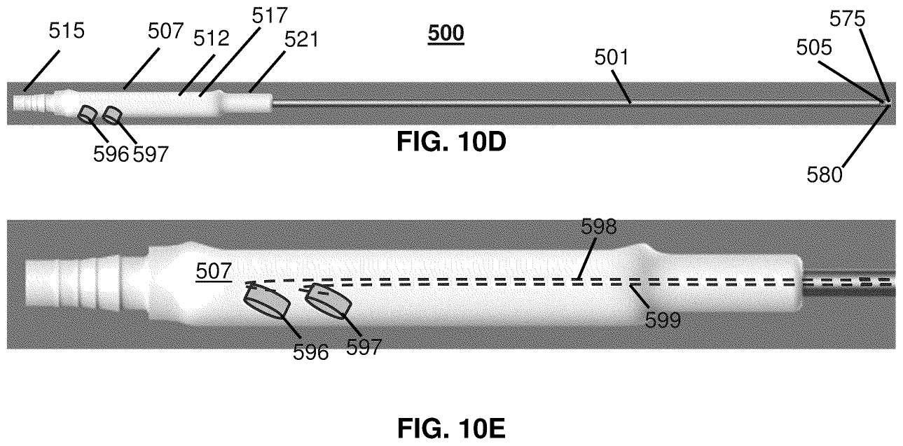

As is described herein, the highly advanced handle can provide multiple functions to each finger of the healthcare provider (or doctor/surgeon). The safer and highly effective technology herein allows for saving lives on a daily basis while empowering a doctor/surgeon. In FIG. 10A is shown a top view of a stylet assembly 500 including a bendable tube 501 and one or more holes 505 at or near the insertion end. The handle 507 has an engagement area (or mating area) 521 capable of engaging with an intubation tube or an endotracheal tube (when the stylet is inserted into the endotracheal tube). The hose fitting 515 can attach to a vacuum hose or suction tubing. The ergonomic finger area 512 has one or more digit or finger holes 517 (only one is shown) that enable vacuum control by the amount of finger covering one or more holes 517 (or by a pressure of the finger or a thumb). In this example, there can be two or more holes 517, and a finger (or thumb, depending on the user) can cover some of the holes to regulate amount of vacuum or can cover all of the holes/none of the holes. Additional powerful capabilities can be provided at optional devices 575 and 580, which can be, for example, camera(s), light(s), cauterizer(s), or any device known in the art. The entire assembly 500 can be controlled and inserted into a patient and an endotracheal tube using one hand (discussed below). In sum, a medical professional can hold the assembly by handle 507 in one hand and have the other hand free to perform additional lifesaving work. FIG. 10B shows an enlarged view of the maneuverable handle illustrating in some embodiments, one or more fins 509 are oriented on the handle 500 to provide instant tactile feedback for proper orientation of the device during use without a user having to look at the device. Each of the one or more fins 509 can be on any side or position on the handle 500. The orientation fin(s) 509 can have any shape such as a triangular shape, semicircular shape, or ergonomic shape. Although not shown, each fin can include a hinge or living hinge at attachment, for example, for flattening when not in use or for actuation control. It should be understood that the one or more fins 509 can be implemented in any of the other figures or embodiments shown herein. According to some aspects, a fin 509 is utilized also as a controlling actuator, for example, for button-like control of 596, 597, or other actuation (depicted in FIG. 10E). FIG. 10C shows an enlarged view of the insertion end with examples of optional features 575 and 580 enlarged.

The terminal functional units (or optional attachments) 575 and 580 can be controlled by various means, including actuators in the handle 507. In FIG. 10C, the terminal functional units 575 and 580 are enlarged to show examples of the capabilities. In this example, the camera unit 575 can be about the size of a grain of salt (average salt is about 300 nm, newatlas.com/salt-sized-disposable-endoscopic-camera/18108/), or about 100 nm to about 5 mm, or about 200 nm to about 1 mm¹. The fiber optic 577, electrical 577, or wireless antennae 577 of the camera 575 can be integrated into the tube 501. The unit 580 can include such example items as a retractable or closeable needle 589, an LED (or other) light source 581, a low temperature or high temperature cauterizer 584, a biopsy sampling tool or forceps 588, or a different accessory attached known in the art. FIG. 10D shows a side view of the assembly 500 and highlights the optional actuators 596 and 597 that can extend/retract/control 575 and 580.

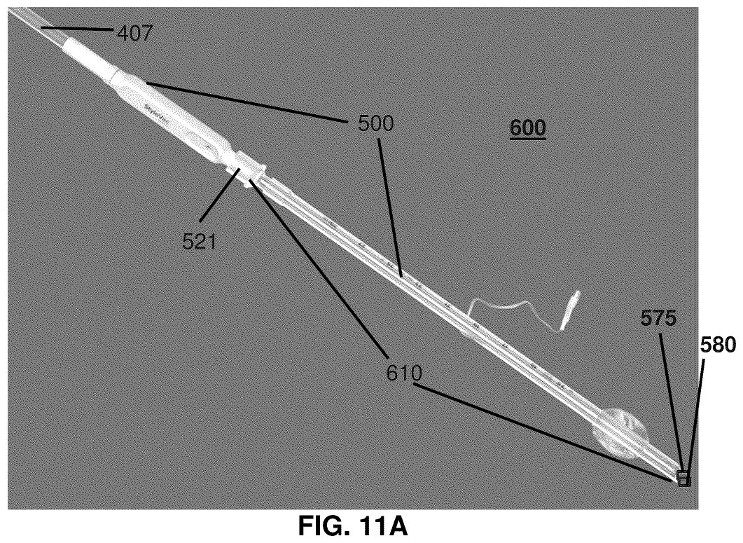

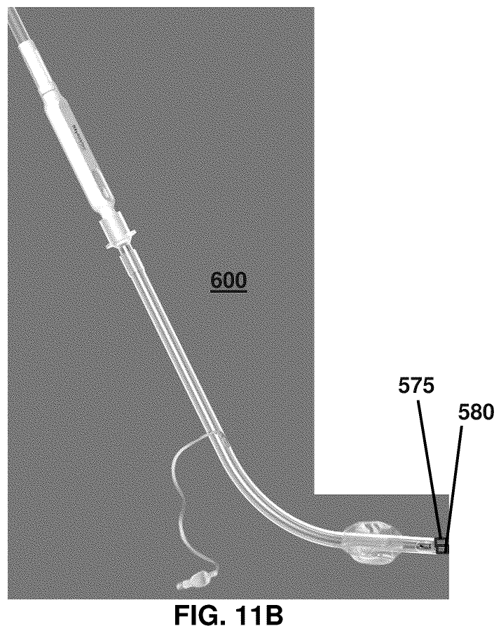

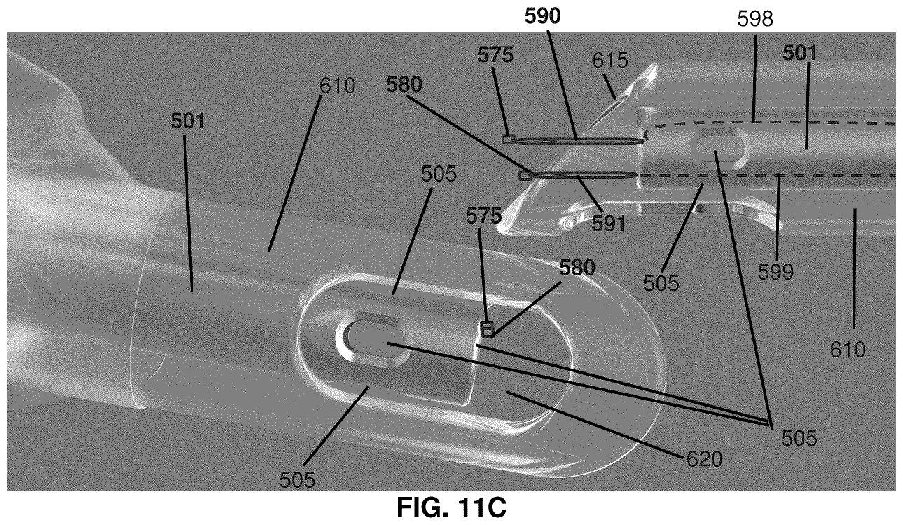

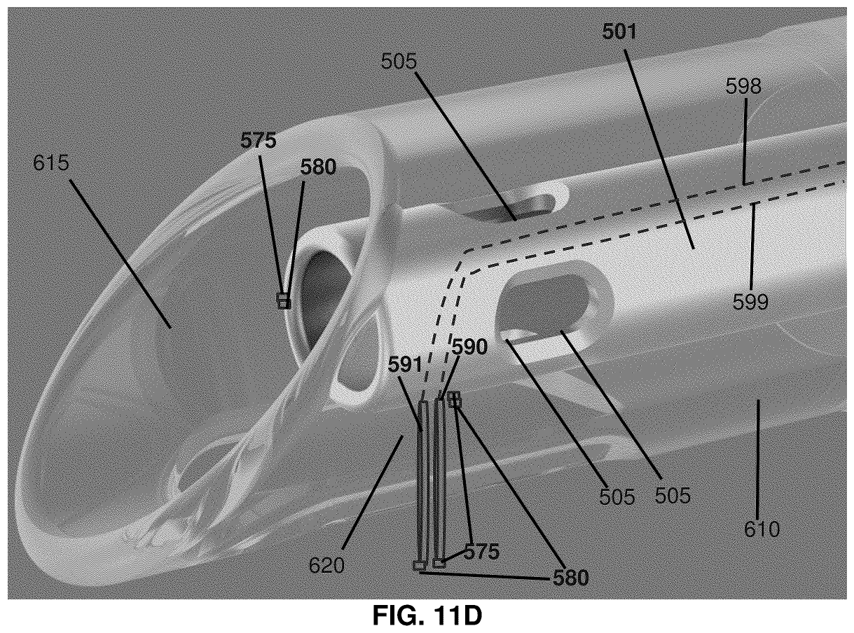

In this example, FIG. 10E shows an enlarged side view of the handle with the optional actuators or controls (e.g., buttons) shown as 596 and 597 connected to optional fluidics, fiber optics, wires (or referred to as “cables”) 598 and 599, respectively. These connections, optical fibers, wires, and/or cables at 598 and 599 can extend all the way to the distal (or insertion) end as will be illustrated in FIG. 11C and FIG. 11D. Turning to an assembled system, FIG. 11A shows the 600, stylet assembly 500 (from FIG. 10A) now inserted into an intubation tube 610 (or endotracheal tube) and engaged with the intubation tube at mating area 521 and ready for suction with a vacuum hose 407 (or suction tubing) attached. In FIG. 11B, the healthcare professional has held handle 507 in one hand and has bent bendable tube 501 to prepare for an intubation.

In more details, turning now to FIG. 11C, two views of the end of an intubation (or endotracheal) tube 610 are shown to illustrate the variable configurations of camera 575 and various units 580. At the murphy eye 620 of the intubation tube 610, the holes (or murphy eyes) 505 of the stylet can be seen, along with an optional terminal hole 505 of the stylet. The camera 575 and the unit 580 (which can be 589, 581, 584, 588, or other from FIG. 10C) can each optionally be attached to an extender 590 and 591, respectively. These optional extenders can be in mechanical or electrical contact with the control wires or cables 598 and 599 and can include such actuators as a solenoid, a lever, or an expanding pressure/filling device. In the case where 598 and 599 include fluidics or pneumatic actuation, a solenoid would not be utilized. It can be seen that each of extender 590 and 591 can be extended into a patient after the entire intubation tube is in place or during a suction. In one or more alternatives (not shown), the units 575 and 580 can each (or one or the other) be attached to the intubation tube 610 instead of to the stylet.

Another example is depicted in FIG. 11D. In this illustration, the extenders 590 and 591 are configured to extend through the murphy eye 620 of the intubation (or endotracheal) tube 610. In this example, the extender 590 and 591 can each or both be configured to be extended by the operator (healthcare professional) after insertion into a patient. The actuators 596 and 597 shown in FIG. 10E are examples of control of these extenders, acting through cable, fiber optic, wire, or fluidic 598 and 599 to reach optional extenders 590 and 591. The camera 575 and additional unit 580 can be extended and remotely controlled.

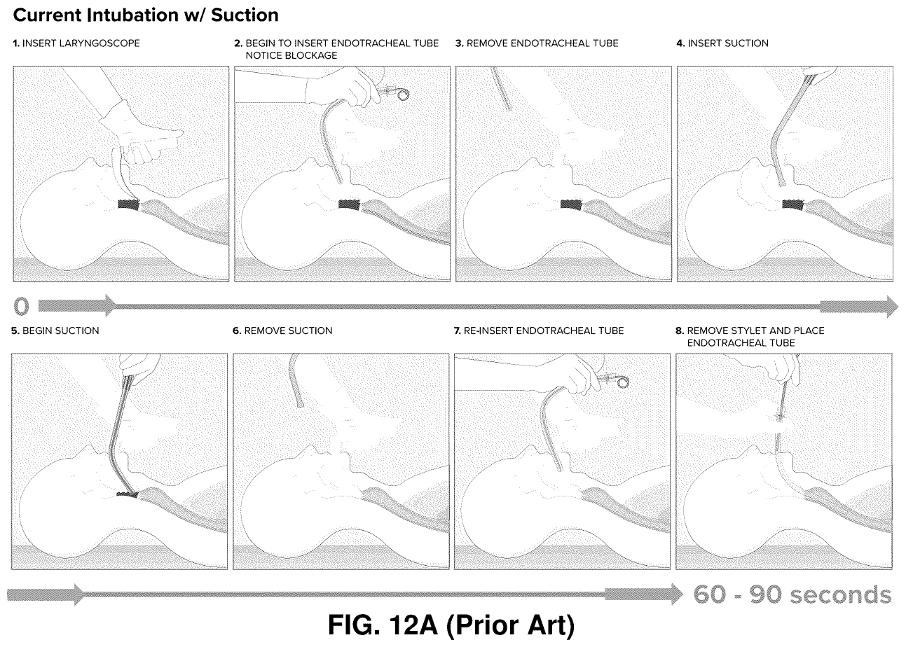

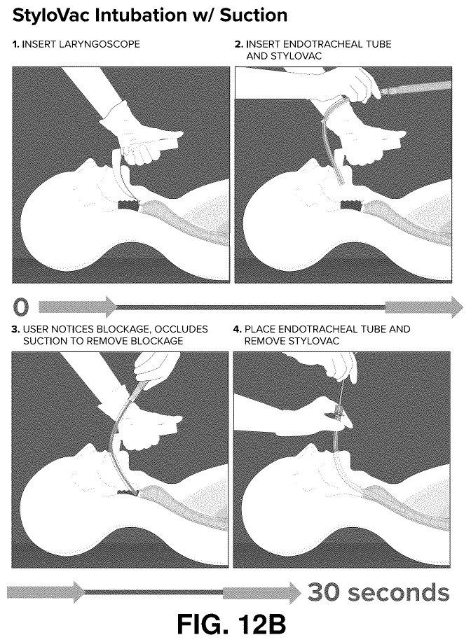

Turning attention now to FIG. 12A (prior art), a currently practiced (most common) intubation procedure with suction is shown, step-by-step. In step 1, a laryngoscope is inserted. In step 2, the healthcare professional has begun to insert the endotracheal tube but has noticed a blockage in the patient. Step 3 shows removal of the endotracheal tube. In step 4, suction is inserted to remove the obstructive fluids. In step 5, suction of the obstructive fluids begins. Suction is removed in step 6. The endotracheal tube is re-inserted in step 7. In step 8, the traditional stylet is removed, and the endotracheal tube is in place. The total time is shown as 60 to 90 seconds but can actually take longer.

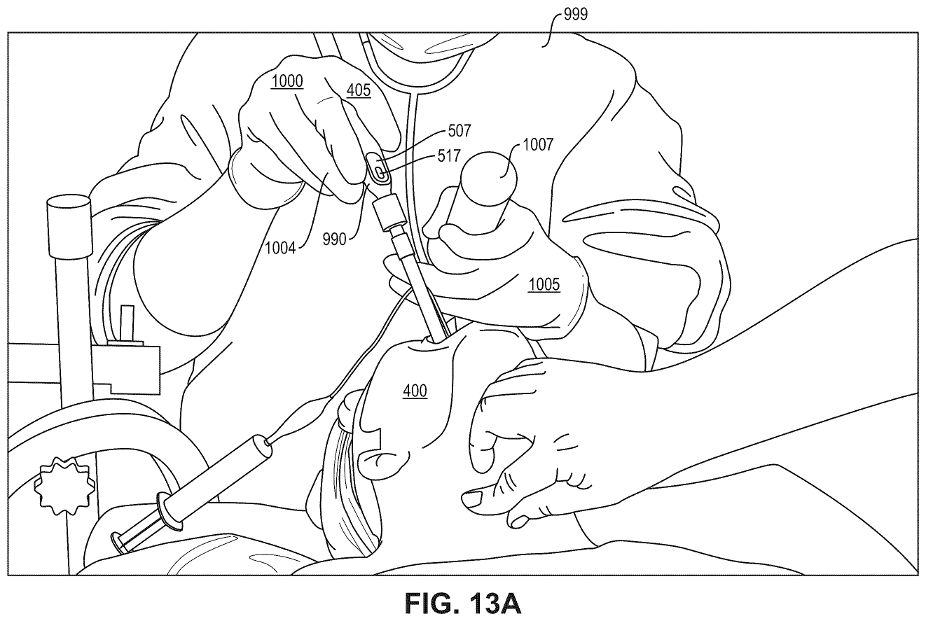

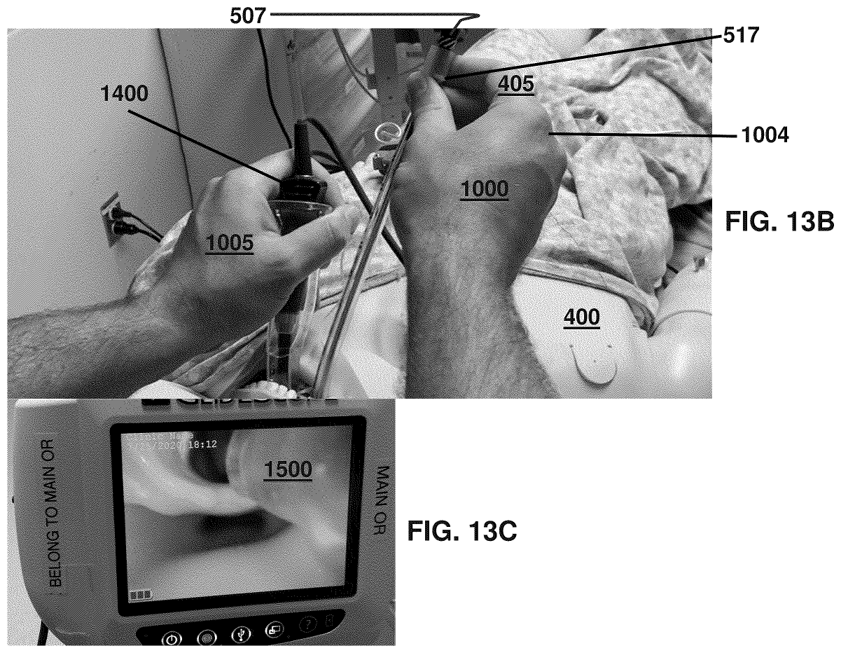

In FIG. 12B, a lifesaving method of the presently disclosed technology is illustrated. In step 1, the laryngoscope is inserted into the patient. In step 2, the endotracheal tube with the stylovac (e.g., of FIGS. 1, 2, 7, 10) is inserted. In step 3, the healthcare professional notices blockage by obstructive fluids and uses a single finger on a single hand to begin suction without having to remove the endotracheal tube (saving precious time). In step 4, the advanced stylet is removed and the endotracheal tube is in place. The total time is shown (for illustration only) as 30 seconds, however the total time is less than the prior art technology and can be less than 90% of the time required in the prior art, can be less than 75% of the time required in the prior art, can be less than 50% of the time required in the prior art, can be less than 40% of the time required in the prior art, can be less than 25% of the time required in the prior art, or optionally can be less than 20% of the time required in the prior art. In a non-limiting example, it can be less than about 30 seconds, can be less than about 2 minutes, can be less than about 5 minutes, or optionally can be less than about 10 minutes. It is clearly seen that the presently disclosed technology can save time, can save complications, and can save lives, and the optional features shown in FIGS. 10C-10E can be simultaneously deployed as in the video and intubation example below (FIGS. 13B-13C).

It is contemplated that in some embodiments, a simultaneous device for providing simultaneous suction and intubation is provided, the simultaneous device comprising: A. a suction stylet assembly for endotracheal intubation, comprising: an elongated tubular member having a first end and a second end, the elongated tubular member having one or more apertures proximate to the first end; a handle member having a first end, a second end, and a side that runs between the first end and the second end, the first end of the handle member in communication with the second end of the elongated tubular member and configured with a stop connector at the communication operative to couple with an endotracheal tube, the handle member including: a first passageway passing through the handle member running from the first end to the second end, the elongated tubular member coaxial with the first passageway; a hose fitting located at the second end, the hose fitting configured to receive a hose that provides suction in removing bodily fluids; one or more apertures located on and about flush with the side, the aperture(s) forming a second passageway through the handle member running between the side and the first passageway, the aperture(s) configured to permit a user to selectively block the aperture(s) with a digit of the user, thereby selectively controlling suction provided between the hose fitting of the handle member and the first end of the elongated tubular member; and B. the endotracheal tube with, at one end, a coupling connector suitable for coupling with the stop connector of the handle member when the elongated tubular member is inserted into the endotracheal tube, to provide the simultaneous device.

According to some aspects, the simultaneous device is capable of providing an insertion into a trachea of a patient and providing simultaneous suction and intubation, and thereafter A can be removed from B, leaving B in position in endotracheal intubation in the patient.

In some embodiments, the simultaneous device is configured wherein the elongated tubular member is a separate piece from the handle member and the elongated tubular member is assembled with the handle member, to combine with the handle member, by an end user prior to a use of the simultaneous device.

According to some aspects, the simultaneous device is configured wherein the handle member is made with the aperture(s) in a recessed position of the handle operable by a single finger of an end user to provide I. a close maneuverability and/or a contact of the handle member in either close contact with a patient, a laryngoscope, or other equipment in use during an intubation and/or a surgery; and II. a single hand control and use of the device by the end user including a single finger control of the suction capability, while freeing a second hand and other fingers of the end user for any other uses.

In some embodiments, the simultaneous device is further comprising a camera disposed at the first end of the elongated tubular member, a light disposed at the first end of the elongated tubular member, a cauterizing/heating filament disposed at the first end of the elongated tubular member, a retractable and/or collapsible needle/disposed at the first end of the elongated tubular member, a biopsy sampling utility or forceps disposed at the first end of the elongated tubular member, or a combination of these thereof.

According to some aspects, the simultaneous device is further comprising one or more buttons or actuators in place on the handle member in reach of fingers that are not above the aperture(s) and each in mechanical, electrical, fluidic, or optical communication with a functional lead (or cable) traversing to the first end of the elongated tubular member through a wall of the member and operative to provide a control at the first end comprising an extension control, an electrical control, a force control, a retraction control, a rotation control, or a combination thereof.

In some embodiments, the simultaneous device is further comprising a camera and a light source disposed at the first end of the tubular member and operative to provide visualization and illumination to the end user during the simultaneous suction and intubation.

According to some aspects, the simultaneous device can provide a simultaneous suction and intubation of a subject in need in less than 30 seconds or in 30 seconds, optionally in less than about 1 minute, optionally in less than about 2 minutes, in less than optionally about 5 minutes, or optionally in less than about 10 minutes. The times are all estimates and are significantly shorter than the prior art device(s)/methods depicted in FIG. 12A. It is contemplated that that an intubation with the presently device could take longer than seconds (e.g., minutes described above) as there could be multiple complicating factors. It should be clear that these times are estimates to show that the presently disclosed device should reduce the time needed to intubate a patient when all else is equal. When compared to previous technology, the presently disclosed devices are far superior in time savings and in preventing other complicating factors.

In some embodiments, the simultaneous device is configured wherein at least one of the one or more apertures of the elongated tubular member is a murphy eye and wherein the murphy eye of the elongated tubular member is concentric with a murphy eye of an endotracheal tube while the elongated tubular member is sheathed by the endotracheal tube.

According to some aspects, a method for performing simultaneous suction and intubation during an insertion of an endotracheal tube into a patient during using a suction stylet assembly for endotracheal intubation is disclosed herein, the method comprising the steps of: (1) obtaining any device disclosed herein, connecting suction tubing to the hose fitting, and bending the device to a suitable shape for an intubation of the patient; (2) using a single hand gripping the handle member, simultaneously inserting the first end of the elongated tubular member and the first end of the endotracheal tube into a trachea of the patient and selectively blocking one or more apertures on the handle member to selectively provide suction to the first end of the elongated tubular member provided by the connected suction tubing, whereby simultaneous suction and intubation is provided during said insertion by a single hand and a single finger; whereby the shape of the handle member permits a close contact (or contact) with the patient, a close maneuverability and/or a contact of the handle member in either close contact with a laryngoscope, or other equipment in use during an intubation and/or a surgery, while freeing a second hand and other fingers of the end user for any other uses; and thereafter removing the suction stylet assembly, A, from the endotracheal tube, B, leaving B in position in endotracheal intubation in the patient.

In some embodiments, the method is wherein selectively blocking the one or more apertures on the handle member is performed with a single finger of the end user.

According to some aspects, the method is executed wherein blocking the one or more apertures on the handle member provides suction that draws unwanted bodily fluids located proximate to the first end of the elongated tubular member, through the elongated tubular member and the handle member, and to the connected suction tubing during the intubation.

In some embodiments, the method is executed wherein the handle member is configured with the aperture(s) in a recessed position of the handle operable by a single finger of an end user to provide the I. a close maneuverability and/or a contact of the handle member in either close contact with a patient, a laryngoscope, or other equipment in use during an intubation and/or a surgery; and II. a single hand control and use of the device by the end user including a single finger control of the suction capability, while freeing a second hand and other fingers of the end user for any other uses.

According to some aspects, the method is executed while further comprising a camera disposed at the first end of the elongated tubular member and/or a light disposed at the first end of the elongated tubular member; and further comprising the step of: (3) actuating the camera and/or the light to visualize inside the patient during said simultaneous suction and intubation.

In some embodiments, the method is executed while further comprising one or more buttons or actuators in place on the handle member in reach of fingers that are not above the aperture(s) and each in mechanical, electrical, fluidic, or optical communication with a functional lead (or cable) traversing to the first end of the elongated tubular member through a wall of the member and operative to provide a control at the first end comprising an extension control, an electrical control, a force control, a retraction control, a rotation control, or a combination thereof; and further comprising the step of: (4) utilizing the one or more buttons or actuators to move one or more accessories at a first end of the tubular member.

According to some aspects, the method is executed while further comprising a biopsy sampling utility or forceps disposed at the first end of the elongated tubular member, or a combination of these; and further comprising the step of: (5) moving said utility or forceps to contact a tissue of the patient and/or to sample a tissue of the patient.

In some embodiments, the method is executed wherein the single hand in step (2) is not prevented from any movement by a physical extension outward from the simultaneous device.

According to some aspects, the method is executed wherein fingers on the hand other than the single finger are free to perform other actions within reach.

In some embodiments, the method is executed in less than 30 seconds or in 30%, 40%, or 50% of the time required to perform an intubation and suction without the device of claim 1. As used herein, the term: “about” means reasonably close to or that a stated number, amount, or value is a reasonable approximation. Any of the numbered items in any of the FIGs. can be combined with any of the numbered items in any other FIG to provide any of the embodiments with the stated goal of saving lives and advancing far beyond the technology depicted in FIG. 12A (prior art).

### EXAMPLES

### Example 1. Simultaneous Suction and Intubation

The technology was designed to provide maximum speed and maneuverability for saving lives. In usage, what is empowered herein is that each hand/finger of the healthcare provider is lifesaving when critical seconds can be saved by the technology and simultaneously when each hand/finger is put to maximum use. In the action photo of FIG. 13A, the healthcare provider 999 (or surgeon) is using one hand 1000 to hold handle 507 and control suction at one or more holes 517 using finger (or digit) 405 while using other fingers 1004 to control other functions; and the other hand 1005 is able to work palpation, a laryngoscope, a camera, a light, computer, or other lifesaving gear while establishing an airway, saving this patient 400. The other fingers and thumb 1004 of hand 1000 are able to operate controls 990 on handle 507. Clearly, the maneuverability of handle 507 allows the surgeon 999 to quickly move the handle at various angles and at close contacts with patient 400, other hand 1005, other gear 1007 (e.g., laryngoscope, light, or lever), while the recessed hole(s) 517 allow vacuum control and other fingers 1004 can provide other controls. These controls can be utilized for camera 575 and/or unit 580, along with one or more extenders 590 and 591, for example. In this example, every second, every finger, and every hand of the surgeon 999 is empowered by the presently disclosed technology. The eyes of the surgeon are illustrated next.

To illustrate actual action from the surgeon's eyes, FIG. 13B shows a view of the patient 400 from the eyes, looking downward, of the surgeon 999 (or healthcare provider). The body of the healthcare provider 999 is not shown in FIG. 13B because the picture is taken from the position of the surgeon 999. The hand 1000 is using finger 405 to control suction at 517 while the other fingers 1004 are able to operate other controls (not visible underneath) of the stylet and the other hand 1005 is simultaneously used to operate a laryngoscope with light 1400. The eyes of the surgeon are empowered during this complex procedure as the camera display 1500 is also visible to the healthcare provider (surgeon), who can perform the procedure with the sleek design of the technology allowing a single hand to perform suction (and other) controls all in close contact with the maneuverable handle 507.

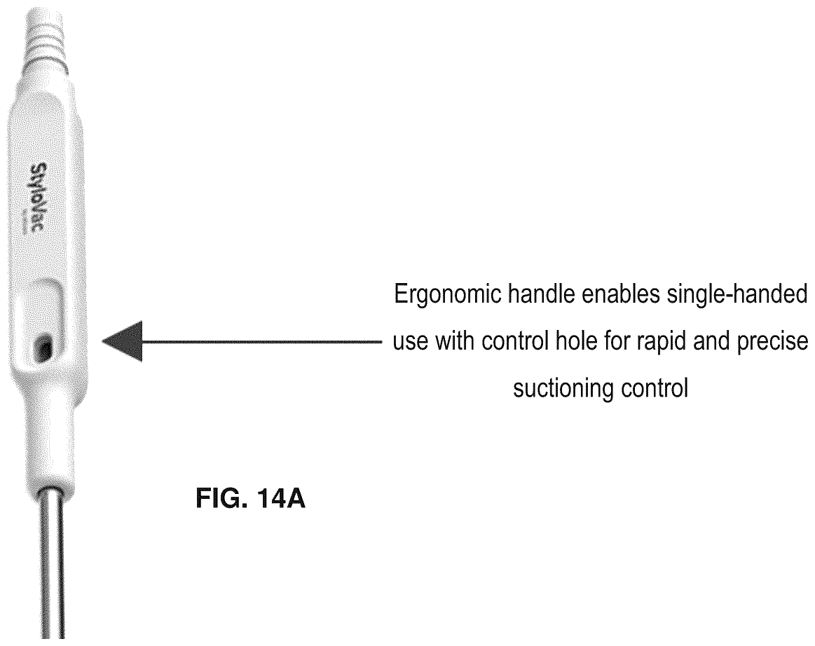

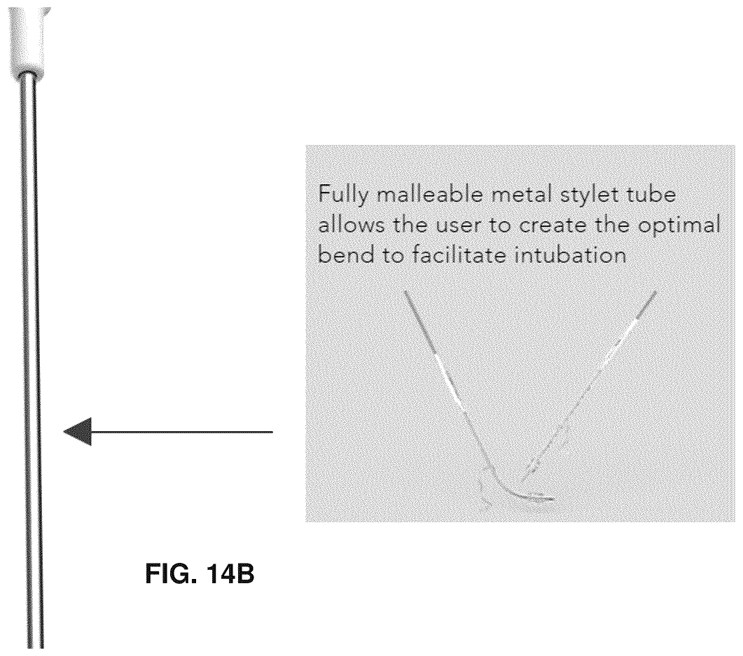

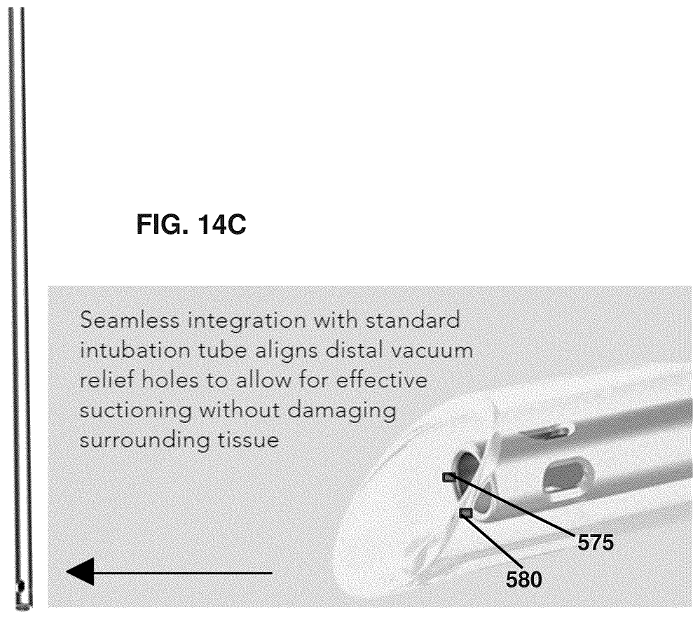

FIG. 14A shows an enlarged view of the ergonomic handle that enables a single hand use with one or more suction control holes sleekly designed in. The photo of a prototype handle of the current technology highlights the maneuverable, single-hand control handle. FIG. 14B shows a photo of a prototype stylet tube of the current technology highlighting the optimum bendability enabled in conjunction with the handle in FIG. 14A. FIG. 14C shows a photo of a prototype distal end of a stylet of the current technology highlighting integration with an endotracheal tube (intubation tube) along with optional features at the end of the stylet. In FIG. 14C the seamless integration with an intubation tube (or endotracheal tube) includes integration of an optional camera 575 and other optional units 580. The optional extending units 590 and 591 are not shown.

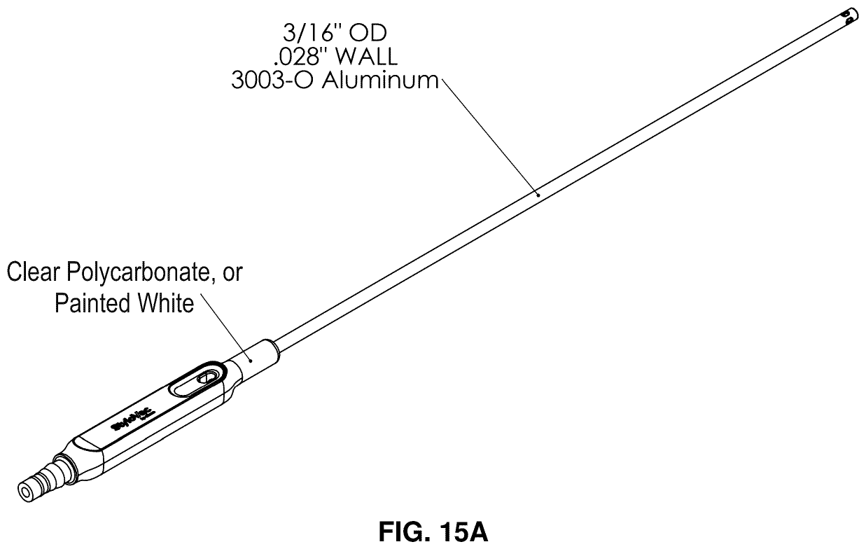

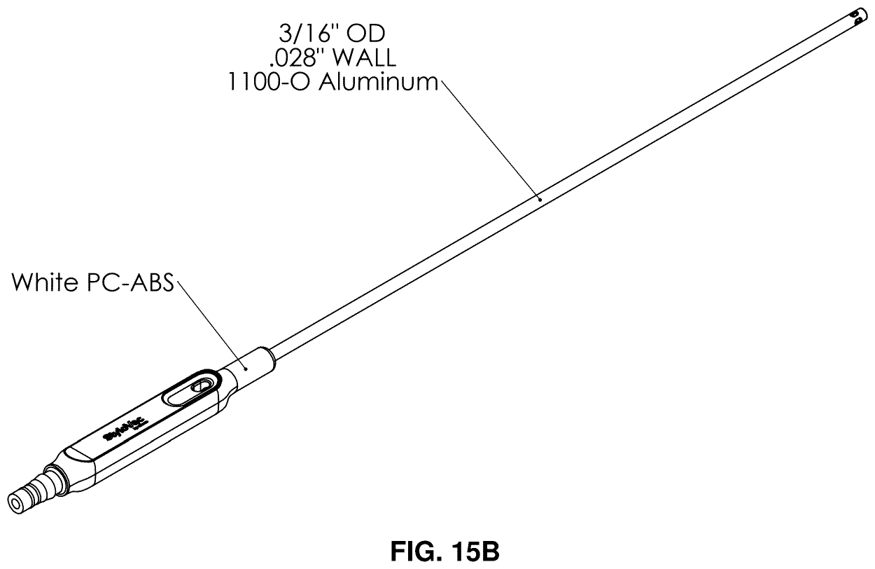

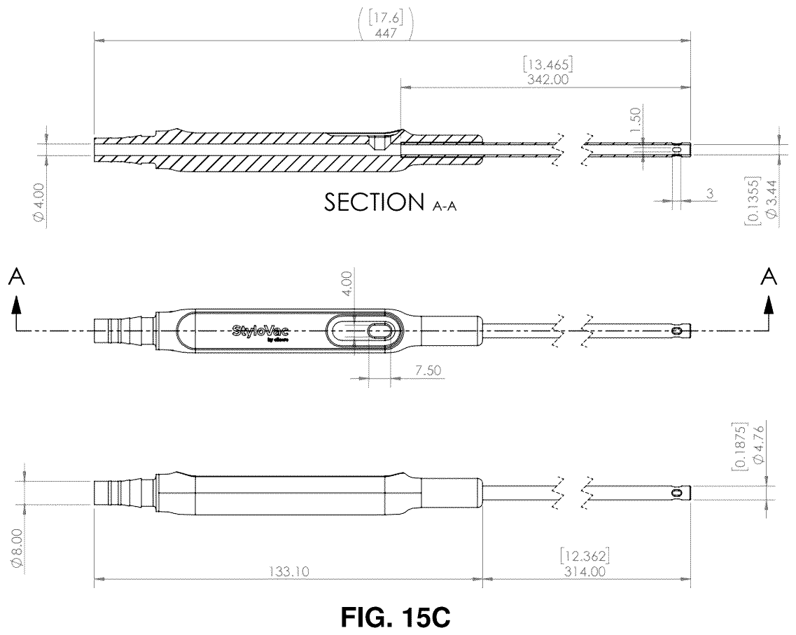

Various prototypes are utilized. FIG. 15A shows a diagram of a prototype (clear or painted) handle of the current technology, highlighting the clear polycarbonate (transparent) option or opaque option along with an example aluminum material stylet tube. This clear design can enable the healthcare provider to see the color of any fluids aspirated through the handle (along with ability to see the bulk or the amounts of fluids traversing through the handle). FIG. 15B shows a diagram of a prototype handle of the current technology highlighting the PC-ABS (polycarbonate/acrylonitrile-butadiene-styrene terpolymer blend) using a thermoplastic alloy of (PC) polycarbonate and (ABS) acrylonitrile-butadiene-styrene prototype construction. FIG. 15C shows mechanical diagrams illustrating example test dimensions of a prototype the current technology highlighting the testing of (single-handed) maneuverability provided by the handle dimensions.

What is claimed is:

1. A simultaneous device for providing simultaneous suction and intubation, the simultaneous device comprising:
A. a suction stylet assembly for endotracheal intubation, comprising:
an elongated tubular member having a first end and a second end, the elongated tubular member having one or more apertures proximate to the first end;
a handle member having a first end, a second end, and a side that runs between the first end and the second end, the first end of the handle member in communication with the second end of the elongated tubular member and configured with a stop connector at the communication operative to couple with an endotracheal tube, the handle member including:
a first passageway passing through the handle member running from the first end to the second end, the elongated tubular member coaxial with the first passageway;
a hose fitting located at the second end, the hose fitting configured to receive a hose that provides suction in removing bodily fluids;
one or more apertures located on and about flush with the side, the aperture(s) forming a second passageway through the handle member running between the side and the first passageway, the aperture(s) configured to permit a user to selectively block the aperture(s) with a digit of the user, thereby selectively controlling suction provided between the hose fitting of the handle member and the first end of the elongated tubular member; and
B. the endotracheal tube with, at one end, a coupling connector suitable for coupling with the stop connector of the handle member when the elongated tubular member is inserted into the endotracheal tube, to provide the simultaneous device;
wherein the simultaneous device is capable of providing an insertion into a trachea of a patient and providing simultaneous suction and intubation, and thereafter A can be removed from B, leaving B in position in endotracheal intubation in the patient.

2. The simultaneous device of claim 1, wherein the elongated tubular member is a separate piece from the handle member and the elongated tubular member is assembled with the handle member, to combine with the handle member, by an end user prior to a use of the simultaneous device.

3. The simultaneous device of claim 1, wherein the handle member is configured with the aperture(s) in a recessed position of the handle operable by a single finger of an end user to provide I. a close maneuverability and/or a contact of the handle member in either close contact with a patient, a laryngoscope, or other equipment in use during an intubation and/or a surgery; and II. a single hand control and use of the device by the end user including a single finger control of the suction capability, while freeing a second hand and other fingers of the end user for any other uses.

4. The simultaneous device of claim 1, further comprising a camera disposed at the first end of the elongated tubular member, a light disposed at the first end of the elongated tubular member, a cauterizing/heating filament disposed at the first end of the elongated tubular member, a retractable and/or collapsible needle/disposed at the first end of the elongated tubular member, a biopsy sampling utility or forceps disposed at the first end of the elongated tubular member, or a combination of these thereof.

5. The simultaneous device of claim 1, further comprising one or more buttons or actuators in place on the handle member in reach of fingers that are not above the aperture(s) and each in mechanical, electrical, fluidic, or optical communication with a functional lead (or cable) traversing to the first end of the elongated tubular member through a wall of the member and operative to provide a control at the first end comprising an extension control, an electrical control, a force control, a retraction control, a rotation control, or a combination thereof.

6. The simultaneous device of claim 1, further comprising a camera and a light source disposed at the first end of the tubular member and operative to provide visualization and illumination to the end user during the simultaneous suction and intubation.

7. The simultaneous device of claim 1 that can provide a simultaneous suction and intubation of a subject in need in less than 50% of a time that is required for a 1) suction and 2) intubation utilizing two or more separate non-simultaneous suction and intubation devices, wherein all other conditions are equal.

8. The simultaneous device of claim 1, wherein at least one of the one or more apertures of the elongated tubular member is a murphy eye and wherein the murphy eye of the elongated tubular member is concentric with a murphy eye of an endotracheal tube while the elongated tubular member is sheathed by the endotracheal tube.

9. A method for performing simultaneous suction and intubation during an insertion of an endotracheal tube into a patient during using a suction stylet assembly for endotracheal intubation, the method comprising the steps of:
1) obtaining the device of claim 1, connecting suction tubing to the hose fitting, and bending the device to a suitable shape for an intubation of the patient;
2) using a single hand gripping the handle member, simultaneously inserting the first end of the elongated tubular member and the first end of the endotracheal tube into a trachea of the patient and selectively blocking one or more apertures on the handle member to selectively provide suction to the first end of the elongated tubular member provided by the connected suction tubing, whereby simultaneous suction and intubation is provided during said insertion by a single hand and a single finger;
whereby the shape of the handle member permits a close contact (or contact) with the patient, a close maneuverability and/or a contact of the handle member in either close contact with a laryngoscope, or other equipment in use during an intubation and/or a surgery, while freeing a second hand and other fingers of the end user for any other uses; and
thereafter removing the suction stylet assembly, A, from the endotracheal tube, B, leaving B in position in endotracheal intubation in the patient.

10. The method of claim 9, wherein selectively blocking the one or more apertures on the handle member is performed with a single finger of the end user.

11. The method of claim 9, wherein blocking the one or more apertures on the handle member provides suction that draws unwanted bodily fluids located proximate to the first end of the elongated tubular member, through the elongated tubular member and the handle member, and to the connected suction tubing during the intubation.

12. The method of claim 9, wherein the handle member is configured with the aperture(s) in a recessed position of the handle operable by a single finger of an end user to provide the I. a close maneuverability and/or a contact of the handle member in either close contact with a patient, a laryngoscope, or other equipment in use during an intubation and/or a surgery; and II. a single hand control and use of the device by the end user including a single finger control of the suction capability, while freeing a second hand and other fingers of the end user for any other uses.

13. The method of claim 9, further comprising a camera disposed at the first end of the elongated tubular member and/or a light disposed at the first end of the elongated tubular member; and further comprising the step of:
(3) actuating the camera and/or the light to visualize inside the patient during said simultaneous suction and intubation.

14. The method of claim 9, further comprising one or more buttons or actuators in place on the handle member in reach of fingers that are not above the aperture(s) and each in mechanical, electrical, fluidic, or optical communication with a functional lead (or cable) traversing to the first end of the elongated tubular member through a wall of the member and operative to provide a control at the first end comprising an extension control, an electrical control, a force control, a retraction control, a rotation control, or a combination thereof; and further comprising the step of:
(4) utilizing the one or more buttons or actuators to move one or more accessories at a first end of the tubular member.

15. The method of claim 9, further comprising a biopsy sampling utility or forceps disposed at the first end of the elongated tubular member, or a combination of these; and further comprising the step of:
(5) moving said utility or forceps to contact a tissue of the patient and/or to sample a tissue of the patient.

16. The method of claim 9, wherein the single hand in step (2) is not prevented from any movement by a physical extension outward from the simultaneous device.

17. The method of claim 9, wherein fingers on the hand other than the single finger are free to perform other actions within reach.

18. The method of claim 9 that can be executed in less than 30 seconds or in 30%, 40%, or 50% of the time required to perform an intubation and suction without the device of claim 1.

19. A kit comprising the simultaneous device of claim 1 and instructions including the method of claim 9 in a form of writing, a video, or both.
